SEQUENCE LISTING (12 sequences)

SEQ ID NO: 1 - DNA, 20 bp, Artificial Sequence
  primer
cagatcgaaggcctgaagga

SEQ ID NO: 2 - DNA, 19 bp, Artificial Sequence
  primer
cttggcccctcagcgtact

SEQ ID NO: 3 - DNA, 19 bp, Artificial Sequence
  primer
ctttgtgctcctcgcttgc

SEQ ID NO: 4 - DNA, 21 bp, Artificial Sequence
  primer
gttatcctggaaaatgcgtgg

SEQ ID NO: 5 - DNA, 20 bp, Artificial Sequence
  primer
ctgaaaagggtccctcggtc

SEQ ID NO: 6 - DNA, 20 bp, Artificial Sequence
  primer
aatagagcgcatgcaaagcc

SEQ ID NO: 7 - DNA, 21 bp, Artificial Sequence
  primer
tcgtttcaaggagcgagattg

SEQ ID NO: 8 - DNA, 20 bp, Artificial Sequence
  primer
ccgctggtgtcttccatgat

SEQ ID NO: 9 - DNA, 22 bp, Artificial Sequence
  primer
ttagccaaacaccttcctgaca

SEQ ID NO: 10 - DNA, 21 bp, Artificial Sequence
  primer
ccgtgacagctgcctctagaa

SEQ ID NO: 11 - DNA, 21 bp, Artificial Sequence
  primer
ccacttcctgcatttcagctt

SEQ ID NO: 12 - DNA, 20 bp, Artificial Sequence
  primer
caccacctccagcaaaaacc

(19) United States
(12) Patent Application Publication
(10) Pub. No.: US 2012/0095301 A1
(43) Pub. Date: Apr. 19, 2012

(54) METHOD OF DETERMINING LYMPH NODE METASTASIS IN CERVICAL CANCER, DEVICE FOR DETERMINING THE SAME, AND COMPUTER PROGRAM

(75) Inventors: Kazuki NAKABAYASHI, Kobe-shi (JP); Kayo HIYAMA, Kobe-shi (JP)

(73) Assignee: SYSMEX CORPORATION, Kobe-shi (JP)

(21) Appl. No.: 13/272,699

(22) Filed: Oct. 13, 2011

(30) Foreign Application Priority Data
Oct. 14, 2010 (JP) 2010-231689

(51) Int. Cl. A61B 5/00

(52) U.S. Cl. 600/300

(57) ABSTRACT

In order to provide a method of testing lymph node metastasis in cervical cancer which can determine the presence of lymph node metastasis in cervical cancer with higher accuracy, the method includes acquiring information about mRNA expression level of at least one gene selected from the group consisting of SERPINB5, CK15, CK4, PI3, and ANXA8 contained in a measurement sample prepared from a lymph node tissue from a cervical cancer patient which may have cervical cancer metastasis; and determining whether cervical cancer has spread to the lymph node tissue based on the information about mRNA expression level acquired.

### FIELD OF THE INVENTION

The present invention relates to a method of determining lymph node metastasis in cervical cancer, a device for determining lymph node metastasis in cervical cancer, and a computer program.

### BACKGROUND

It is known that cancer cells in a cancer patient's body flow through blood or lymph, move from a primary tumor, and grow at other sites (metastasis). Particularly, a state that the cancer cells have reached lymph nodes by the flow of lymph and grown therein is called lymph node metastasis.

In cervical cancer, lymph nodes are tissues in which the cancer tends to metastasize at a high rate. Thus, not only cancer but also lymph nodes are usually resected in surgery of cervical cancer.

However, sequelae such as lymphedema are often caused by the resection of lymph nodes after surgery. Further, autonomic nerves may be cut when resecting the lymph nodes. Consequently, the problem is that serious sequela such as dysuria and dyschezia can be caused after surgery.

A study of diagnosis of lymph node metastasis for detecting cancer cells in lymph nodes has been recently made. For example, a process of diagnosing lymph node metastasis in cervical cancer which includes determining whether the cancer cells are present in a sentinel lymph node (SLN) at which cancer cells first arrive from the primary tissue is discussed. If the spread of cervical cancer to lymph nodes is not found by the diagnosis, resection of the lymph nodes is not required. This leads to an improvement in postoperative patient's QOL.

Thus, in the diagnosis of cervical cancer, the presence of lymph node metastasis is a useful information to determine a therapeutic strategy, such as the extent of resection in surgery. Since the diagnosis of lymph node metastasis in cervical cancer can be used to accurately determine the stage of cancer progression and its importance is increasing.

In many medical institutions, an examination of the presence of lymph node metastasis is currently performed by using tissue diagnosis in which a section prepared from a lymph node tissue is observed with a microscope.

However, the tissue diagnosis may not provide a result of accurate diagnosis when a section prepared with a cut surface not containing cancer cells is used or when lymph node metastasis is micrometastasis, even in the presence of the cancer cells in the lymph node tissue. Further, a result of the tissue diagnosis may vary depending on the skill of a pathologist in charge of the tissue diagnosis.

Thus, a method of examining lymph node metastasis in cervical cancer based on the expression of a molecular marker is proposed. For example, molecular markers of lymph node metastasis in cervical cancer are reported in the article (Wang H. Y. et al. Int J Gynecol Cancer, vol. 16, p. 643-648 (2006)) and the article (Samouelian V. et al. Int J Bio Markers, vol. 23, p. 74-82 (2008)). Specifically, the article of Wang H. Y. et al. describes that the CK19 is a molecular marker useful in identifying micrometastases as a result of RT-PCR of SLN or immunohistochemistry (IHC) analysis. The article of Samouelian V. et al. describes that CK19, MUC1, and the like are useful as molecular markers from the results of RT-PCR in uterine cervix cancer tissues and lymph nodes negative for metastasis.

As described above, the diagnosis of lymph node metastasis in cervical cancer gives a large effect on patient's QOL and is used to determine the extent of resection in surgery. Accordingly, high accuracy is required for the diagnostic result. In the diagnosis of lymph node metastasis using a molecular marker, as a matter of fact, the accuracy of the diagnostic result is improved as the number of a target molecular marker is increased. For example, when diagnostic results obtained by using CK19 and MUC1 (known molecular markers) are false-positive or false-negative, determination of more appropriate therapeutic strategies is achieved by referring to other molecular markers. Thus, there has been a need for development of a novel molecular marker useful for the diagnosis of lymph node metastasis in cervical cancer.

Relationships among serpin peptidase inhibitor, clade B member 5 (SERPINB5), cytokeratin 15 (CK15), cytokeratin 4 (CK4), peptidase inhibitor 3 (PI3), and annexin A8 (ANXA8) and various types of cancers are reported.

The article (Domann F. E. et al. Int. J. Cancer, vol. 85, p. 805-810 (2000)) reports that maspin (another name for SERPINB5) is an antioncogene being down-regulated in breast cancer. The U.S. patent literatures (US2010/047771 and US2006/078941) show that the maspin gene is highly expressed in serous papillary adenocarcinoma which is a type of lung cancer or ovarian cancer rather than a normal tissue. The article (Karanjawala, Z. E. et al. Am J Surg Pathol., vol. 32, p. 188-196 (2008)) reports that ANXA8 is expressed in invasive pancreatic ductal adenocarcinoma. The U.S. patent literature (US2007/172857) discloses that a difference in the expression level between the lymph nodes positive or negative for metastasis is found to be small as a result of the research of the expression level of ANXA8 in tissues of lymph nodes positive or negative for metastasis of colon cancer. However, there is no description regarding cervical cancer in these articles. As a matter of fact, there is neither description nor suggestion about relationships among lymph nodes positive for metastasis of cervical cancer and expression levels of SERPINB5 and ANXA8.

The article (Pyeon D. Cancer Res, vol. 67, p. 4605-4619 (2007)) reports that expression of genes such as KRT4 (another name for CK4) and KRT15 (another name for CK15) is down-regulated in the primary tumor of cervical cancer as compared with a normal tissue. Although specific data is not shown, the article (Clauss A. et al. Neoplasia, vol. 12, and p. 161-172 (2010)) describes that elafin (another name for PI3) is expressed in a uterine cervix cancer cell line (Hela). Here, in the art, it is known that established cells are different in cell characteristics, such as gene expression patterns, from cancer cells which are actually extracted from patients in many cases. However, neither description nor suggestion regarding relationships among lymph nodes positive for metastasis of cervical cancer and expression levels of CK4, CK15, and PI3 is found in all the articles.

As described above, there are still few reports of the molecular marker related to lymph node metastasis in cervical cancer. Thus, further development of a novel molecular marker useful in determining lymph node metastasis in cervical cancer and a method of determining lymph node metastasis using the marker is desired.

### SUMMARY OF THE INVENTION

The scope of the present invention is defined solely by the appended claims, and is not affected to any degree by the statements within this summary.

An object of the present invention is to provide a method of determining lymph node metastasis in cervical cancer using a novel molecular marker which can determine lymph node metastasis in cervical cancer. Further, an object of the present invention is to provide a device for determining lymph node metastasis in cervical cancer and a computer program.

The present inventors have examined mRNA expression profiles in lymph nodes in which metastasis of cervical cancer is histologically found (hereinafter also referred to as “lymph nodes positive for metastasis”) and lymph nodes in which metastasis of cervical cancer is not histologically found (hereinafter also referred to as “lymph nodes negative for metastasis”).

As a result, the present inventors have found that lymph nodes positive for metastasis can be distinguished from lymph nodes negative for metastasis with high accuracy based on the analysis results of mRNA expression levels of Serpin peptidase inhibitor, clade B member 5 (SERPINB5), Cytokeratin 15 (CK15), Cytokeratin 4 (CK4), Peptidase inhibitor 3 (PI3), and Annexin 8 (ANXA8), and the present invention has been completed.

A first aspect of the present invention is a method of determining lymph node metastasis in cervical cancer, comprising:

acquiring information about mRNA expression level of at least one gene selected from the group consisting of SERPINB5, CK15, CK4, PI3, and ANXA8 contained in a measurement sample prepared from a lymph node tissue from a cervical cancer patient which may have cervical cancer metastasis; and

determining whether cervical cancer has spread to the lymph node tissue based on the acquired information about mRNA expression level.

A second aspect of the present invention is a device for determining lymph node metastasis in cervical cancer, comprising:

a measurement unit which acquires information about mRNA expression level of at least one gene selected from the group consisting of SERPINB5, CK15, CK4, PI3, and ANXA8 contained in a measurement sample prepared from a lymph node tissue which may have cervical cancer metastasis;

a determination unit which determines whether cervical cancer has spread to the lymph node tissue based on the information about mRNA expression level acquired by the measurement unit; and

an output unit which outputs the determination result obtained by the determination unit.

A third aspect of the present invention is a computer program product comprising: a computer readable medium; and software instructions on the computer readable medium, for enabling the computer to perform predetermined operations comprising:

receiving information about mRNA expression level of at least one gene selected from the group consisting of SERPINB5, CK15, CK4, P13, and ANXA8 contained in a measurement sample prepared from a lymph node tissue which may have cervical cancer metastasis;

determining whether cervical cancer has spread to the lymph node tissue based on the information about mRNA expression level received in the receiving step; and

outputting the determination result obtained by the determining process.

According to the method of determining lymph node metastasis in cervical cancer, the device for determining lymph node metastasis in cervical cancer, and the computer program in the present invention, the presence of lymph node metastasis in cervical cancer can be examined with higher accuracy.

### BRIEF DESCRIPTION OF THE DRAWINGS

FIG. 1 shows an embodiment of a determining device which executes determination of lymph node metastasis in cervical cancer;

FIG. 2 is a flow chart showing an example of determination of lymph node metastasis in cervical cancer; and

FIG. 3 is a graph showing a relationship between the presence of lymph node metastasis as for each gene and the cycle number of PCR.

### DETAILED DESCRIPTION OF THE PREFERRED EMBODIMENTS

The preferred embodiments of the present invention will be described hereinafter with reference to the drawings.

[1. Method of Determining Lymph Node Metastasis in Cervical Cancer]

The method of determining lymph node metastasis in cervical cancer of the present invention (hereinafter simply referred to as “a determination method”) includes acquiring information about mRNA expression level of at least one gene selected from the group consisting of SERPINB5, CK15, CK4, PI3, and ANXA8 contained in a measurement sample prepared from a lymph node tissue from a cervical cancer patient which may have cervical cancer metastasis; and determining whether cervical cancer has spread to the lymph node tissue of the patient based on the information about the mRNA expression level.

The base sequences of mRNAs of the above genes are available from the GenBank database provided by National Center for Biotechnology Information. The GenBank accession numbers of each gene are as shown in Table 1 below. The GenBank accession numbers indicate the most recent number as of Oct. 12, 2010.

TABLE 1

| Gene symbol | Accession number |
|---|---|
| SERPINB5 | NM_002639 |
| CK15 | NM_002275 |
| CK4 | NM_002272 |
| PI3 | NM_002638 |
| ANXA8 | NM_001040084 |

[1-1. Acquisition of Information About Expression Level of mRNAs]

In the determination method of the present invention, information about mRNA expression level of at least one gene selected from the group consisting of SERPINB5, CK15, CK4, PI3, and ANXA8 contained in a measurement sample prepared from a lymph node tissue from a cervical cancer patient which may have cervical cancer metastasis is first acquired.

The gene from which the information about mRNA expression level is acquired may be one of the five genes. In order to improve the determination accuracy in the subsequent determining step, it is preferable to acquire information about mRNA expression levels of multiple genes.

The lymph node tissue is not particularly limited as long as it is a tissue including a lymph node in the pelvis. Examples of the lymph node include common iliac lymph nodes, external iliac lymph nodes, internal iliac lymph nodes, middle rectal root lymph nodes, and obturator lymph nodes. These lymph nodes may be suitably selected depending on the condition of patients being subjected to the determination method according to the present embodiment.

The measurement sample to be used for the method of the present invention is not particularly limited as long as it is a sample which contains mRNAs of the five genes. Preparation of such a measurement sample may be performed by known methods in the art. For example, the measurement sample containing RNAs can be obtained by subjecting a lymph node tissue in a suitable pretreatment liquid to a physical process (stirring, homogenization, ultrasonic fragmentation or the like) and allowing RNAs contained in cells in the tissue to be released in a solution. In the process of preparing the measurement sample, the measurement sample is prepared simply in a short time, it is preferably performed by a method using the pretreatment liquid.

As for the measurement sample obtained in the above manner, a residue of the tissue or cells may be removed by a known method in the art, such as centrifugation, filtering or column chromatography, if necessary. RNAs contained in the measurement sample may be purified. For example, RNAs in the measurement sample can be purified by centrifuging a solution (measurement sample) containing RNAs released from cells is centrifuged, recovering a supernatant, and extracting the supernatant by a phenol/chloroform procedure.

Preparation and purification of RNAs in the measurement sample from the lymph node tissue may be performed using commercially available RNA extraction/purification kits.

The pretreatment liquid to be used for preparation of the measurement sample is not particularly limited as long as it can be used for extraction of mRNAs from cells contained in the lymph node tissue. It is preferable that the pretreatment liquid has an acid pH in order to avoid degradation of RNA. Such a pH is preferably from pH 2.5 to pH 5.0, more preferably from pH 3.0 to pH 4.0. As the pretreatment liquid, a solution containing a buffer such as a glycine chloride buffer is listed. The concentration of the buffer in the pretreatment liquid may be suitably set within a range which keeps the pH of the pretreatment liquid acidic.

It is preferable that the pretreatment liquid further contains a surfactant in order to allow mRNAs to be efficiently extracted from cells contained in the lymph node tissue. The surfactant is not particularly limited as long as it is capable of damaging cell membranes and nuclear membranes of cells contained in the lymph node tissue and allowing nucleic acids in the cells to be released in the Solution. Examples of the surfactant include nonionic surfactants. As for the nonionic surfactants, a polyoxyethylene-based nonionic surfactant is preferred and a polyoxyethylene-based nonionic surfactant represented by Formula (I) is more preferred.

R¹—R²—(CH₂CH₂O)ₙ—H (I)

(wherein R¹ represents an alkyl group having 10 to 22 carbon atoms, an alkenyl group having 10 to 22 carbon atoms, an alkynyl group having 10 to 22 carbon atoms or an isooctyl group having 10 to 22 carbon atoms, R² represents an oxygen atom or a phenyleneoxy group, and n represents an integer of 8 to 120).

The polyoxyethylene-based nonionic surfactant is not particularly limited. Examples thereof include polyoxyethylene lauryl ether, polyoxyethylene cetyl ether, polyoxyethylene oleyl ether, polyoxyethylene myristyl ether, polyoxyethylene stearyl ether, polyoxyethylene nonylphenyl ether, and polyoxyethylene isooctyl phenyl ether. Among them, polyoxyethylene lauryl ether (addition mole number of an oxyethylene group: 23) (trade name: Brij (registered trademark) 35), manufactured by Sigma Aldrich) or the like may be used as the polyoxyethylene-based nonionic surfactant. The concentration of the surfactant in the pretreatment liquid is preferably from 0.1 to 6% by volume, more preferably from 1 to 5% by volume from the viewpoint of sufficiently extracting mRNA from cells contained in the lymph node tissue.

When the information about mRNA expression level of the gene is acquired by a method of amplifying nucleic acid to be described later, it is preferable that the pretreatment liquid contains dimethyl sulfoxide (DMSO). This allows a decrease in the activity of a nucleic acid synthesis enzyme which is used for the method of amplifying nucleic acid to be controlled. Further, even if a substance (inhibitor) which inhibits the reaction of the nucleic acid synthesis enzyme is contained in the lymph node tissue, the influence of the inhibitor may be effectively reduced. The concentration of DMSO in the pretreatment liquid is preferably from 1 to 50% by volume, more preferably 5 to 30% by volume, still more preferably from 10 to 25% by volume from the viewpoint of sufficiently exhibiting the effect.

The amount of the pretreatment liquid to be used for preparation of the measurement sample can be suitably set depending on the type of the pretreatment liquid. Usually, it is from 6 to 80 μL per 1 mg of lymph node tissue. A mixing process of cells contained in the lymph node tissue and the pretreatment liquid is performed by, for example, stirring the cells contained in the lymph node tissue and the pretreatment liquid at room temperature.

In the determination method of the present invention, the information about the mRNA expression level of genes of SERPINB5, CK15, CK4, PI3, and ANXA8 can be acquired by known methods in the art, such as the method of amplifying nucleic acid. The information about the mRNA expression level can be acquired by a microarray hybridization method using microarrays in which nucleic acid probes complementary to base sequences of the genes are arranged. Among them, the method of amplifying nucleic acid to be described later is preferable to acquire the information about the mRNA expression level.

In the present specification, the “information about mRNA expression level of gene” is not particularly limited as long as it is data which shows mRNA expression level of genes of SERPINB5, CK15, CK4, PI3, and ANXA8.

Examples of the data when the data is acquired by the method of amplifying nucleic acid include optical measurement values of the reaction solution after amplification (e.g. fluorescence intensity, turbidity, and absorbance). The data showing the expression level of mRNA includes an optical measurement value based on the amplified mRNA, (the cycle number) or time when the optical measurement value reaches a predetermined standard value, the cycle number (or time) when the variation of the optical measurement value reaches a predetermined standard value, and the quantitative value of mRNA calculated from the optical measurement value (or the cycle number) and the calibration curve in the nucleic acid amplifying method.

Here, the predetermined standard value may be suitably set according to the type of the data. For example, when the data is the cycle number, the variation of fluorescence intensity when the nucleic acid amplification reaction shows a logarithmic growth may be set as a predetermined standard value.

When the data showing the mRNA expression levels of the genes is acquired using the microarrays, an example of the data includes a signal strength (a change in fluorescence intensity, coloring intensity, and current amount) derived from hybridization among nucleic acid probes on the microarrays and mRNAs of the genes of SERPINB5, CK15, CK4, P13, and ANXA8 or nucleic acids derived from the mRNAs (cDNA, cRNA and the like). The data includes a quantitative value of mRNA which is acquired from the signal strength and the calibration curve.

The method of amplifying nucleic acid can be performed by maintaining a reaction solution that contains a prepared measurement sample and a primer for amplifying mRNA of at least one gene selected from SERPINB5, CK15, CK4, PI3, and ANXA8 or a nucleic acid derived from the mRNA (cDNA, cRNA or the like), and a nucleic acid synthesis enzyme in a suitable reaction condition.

Examples of the method of amplifying nucleic acid which is suitable for the determination method of the present invention include a polymerase-chain-reaction assay (PCR), a strand displacement activity assay, a ligase-chain-reaction assay, and a transcriptional amplification method. These nucleic acid amplifying methods themselves are all known methods in the art.

As the PCR assay, for example, a real time RT-PCR (Reverse Transcription-PCR) assay is listed. As the strand displacement activity assay, for example, a real-time RT-LAMP (Reverse Transcription-LAMP) assay (refer to, for example, U.S. Pat. No. 6,410,278) is listed. As the transcriptional amplification method, for example, a TAS (Transcription-based Amplification System) assay is listed. Among these methods, the real-time RT-PCR assay and the real-time RT-LAMP assay are preferred from the viewpoint of easy acquisition of the data.

Examples of the real-time RT-PCR assay include a TaqMan method (“TaqMan” is a registered trademark of Roche Molecular Systems Inc.) and an intercalator method using an intercalator (SYBR Green, manufactured by Molecular Probe Inc.). In the real-time RT-PCR assay, an optical state (fluorescence intensity, coloring intensity and the like) of the reaction solution varies with the nucleic acid amplification. Thus, the information about mRNA expression levels of the genes can be acquired based on the optical measurement value of the reaction solution or the cycle number until the value reaches a predetermined standard value.

In the real-time RT-LAMP assay, an insoluble magnesium pyrophosphate byproduct is produced by the amplification of cDNA derived from mRNA. The reaction solution becomes cloudy [symbol] the magnesium pyrophosphate is increased. Therefore, in the real-time RT-LAMP assay, the information about mRNA expression levels of the genes can be acquired based on the turbidity and absorbance measured values of the reaction solution or the time until these values reach predetermined standard values.

The primer to be used for the method of amplifying nucleic acid is not particularly limited as long as it can amplify mRNAs of the genes of SERPINB5, CK15, CK4, PI3, and ANXA8 or cDNAs derived from the mRNAs. Such a primer may be designed based on base sequences of mRNAs of the genes or cDNAs derived from the mRNAs. It is preferable that the primer is designed according to the type of the method of amplifying nucleic acid. The length of the primer is usually from 5 to 50 nucleotides, preferably from 10 to 40 nucleotides. The primer itself can be produced by any nucleic acid synthesis known in the art.

The primer may be used as a primer pair or a primer set which” can amplify mRNAs of the genes or cDNAs derived from the mRNAs.

The primer may be modified by techniques usually used in the art. Labeling of the primer may be performed using radioactive elements or non-radioactive molecules. Examples of the radioactive isotope include ³²P, ³³P, ³⁵S, ³H, and ¹²⁵I. Examples of the nonradioactive substance include ligands such as biotin, avidin, streptoavidin, and digoxigenin; hapten, pigment, and luminescent reagents such as chemiluminescent, bioluminescent, fluorescent and phosphorescent reagents.

As the nucleic acid synthesis enzyme, enzymes according to the type of method of amplifying nucleic acid may be used. Examples of the nucleic acid synthesis enzyme include reverse transcriptase, DNA polymerase, strand-displacement DNA polymerase, and ligase. When the nucleic acid amplifying method is the real-time RT-PCR assay, reverse transcriptase and DNA polymerase are used as the nucleic acid synthesis enzyme.

Examples of the reverse transcriptase include Avian Myeloblastosis Virus (AMV) reverse transcriptase and (Molony Murine Leukemia Virus (M-MLV) reverse transcriptase. Examples of the DNA polymerase include Taq DNA polymerase, Pfu DNA polymerase, T4 DNA polymerase, and Bst DNA polymerase. In the real-time RT-PCR assay, an enzyme having both reverse transcriptase activity and DNA synthesis activity may be used as the nucleic acid synthesis enzyme.

The reaction condition for the method of amplifying nucleic acid varies depending on the type of the method of amplifying nucleic acid and the sequence of the primer and it can be set with reference to, for example, a method described in (Molecular Cloning: A Laboratory Manual (2nd ed.) (Sambrook, J. et al. Cold Spring Harbor Laboratory Press, New York (1989)).

[1-2. Determination of Lymph Node Metastasis in Cervical Cancer]

In the determination method of the present invention, it is determined whether cervical cancer has spread to the lymph node tissue from the cervical cancer patient which may have cervical cancer metastasis based on the acquired information about mRNA expression levels of the genes.

As described above, mRNA expression levels of the genes of SERPINB5, CK15, CK4, PI3, and ANXA8 are found largely in lymph nodes positive for metastasis as compared with those of lymph nodes negative for metastasis, which is found out by the present inventors. Therefore, when the mRNA expression level is overexpressed, it is determined that cervical cancer has spread to the lymph node tissue. For example, in the case where the information about mRNA expression level include optical measurement values (fluorescence intensity, turbidity, and absorbance) of a reaction solution after a sufficient reaction time for amplification of nucleic acid in the method of amplifying nucleic acid, when the value is large, it is determined that cervical cancer has spread to the lymph node tissue from the cervical cancer patient. On the contrary, when the measured value is small, it is determined that cervical cancer has not spread to the lymph node tissue.

The determination of lymph node metastasis by the measured values themselves may be experientially performed using accumulated data. However, in the case of micrometastasis, the determination with higher accuracy may be required. In that case, it is preferably determined whether the mRNA expression levels of the genes are overexpressed based on compared results obtained by comparing data about the expression level with a threshold value according to the type of the data. For example, in the case where the data is the cycle number (or time) when the variation of the optical measurement value (fluorescence intensity, turbidity, absorbance or the like) of the reaction solution after nucleic acid amplification reaches a predetermined standard value, it is determined that the acquired mRNA expression level is overexpressed when the cycle number (or time) is not more than the predetermined threshold value. Therefore, in this case, it is determined that cervical cancer has spread to the lymph node tissue.

When the cycle number (or time) is larger than the predetermined threshold value, it is determined that the acquired mRNA expression level is not overexpressed. Therefore, in this case, it is determined that cervical cancer has not spread to the lymph node tissue.

In the case where the information about mRNA expression levels of the genes is quantitative values of the mRNA levels, it is determined that the mRNA expression levels are overexpressed when the quantitative values are more than predetermined threshold values. Therefore, in this case, it is determined that cervical cancer has spread to the lymph node tissue.

When the quantitative values of mRNAs are smaller than the predetermined threshold values, it is determined that the mRNA expression levels are not overexpressed. Therefore, in this case, it is determined that cervical cancer has not spread to the lymph node tissue.

The “predetermined threshold value(s)” to be used for the determination method of the present invention may be set according to the type of the data about the expression level. That is, the threshold value may be experientially set to a value having a predetermined expression level which may clearly distinguish between a lymph node group in which cervical cancer metastasis is observed and a lymph node group in which lung cancer metastasis is not observed. Specifically, when the data about expression level is the cycle number, the cycle numbers of multiple lymph nodes in which lymph nodes in which metastasis of cervical cancer is histologically found and multiple lymph nodes in which metastasis of cervical cancer is not histologically found are acquired. The cycle numbers that can distinguish between a group of the lymph nodes in which lymph node metastasis is found and a group of the lymph nodes in which lymph node metastasis is not found can be set as predetermined threshold values.

[2. Device for Determining Lymph Node Metastasis in Cervical Cancer]

Subsequently, the device for determining lymph node metastasis in cervical cancer according to the embodiment of the present invention (hereinafter simply referred to as “a determination device”) will be described with reference to the drawings.

The determination device according to the present embodiment is an example of a suitable apparatus to perform the determination method according to the embodiment of the present invention. The determination device according to the present embodiment is a device that performs the determination by employing the cycle number when the variation of fluorescence intensity reaches the predetermined standard value as the information about mRNA expression level and comparing the cycle number with the threshold value about the cycle number.

[2-1. Configuration of Determining Device]

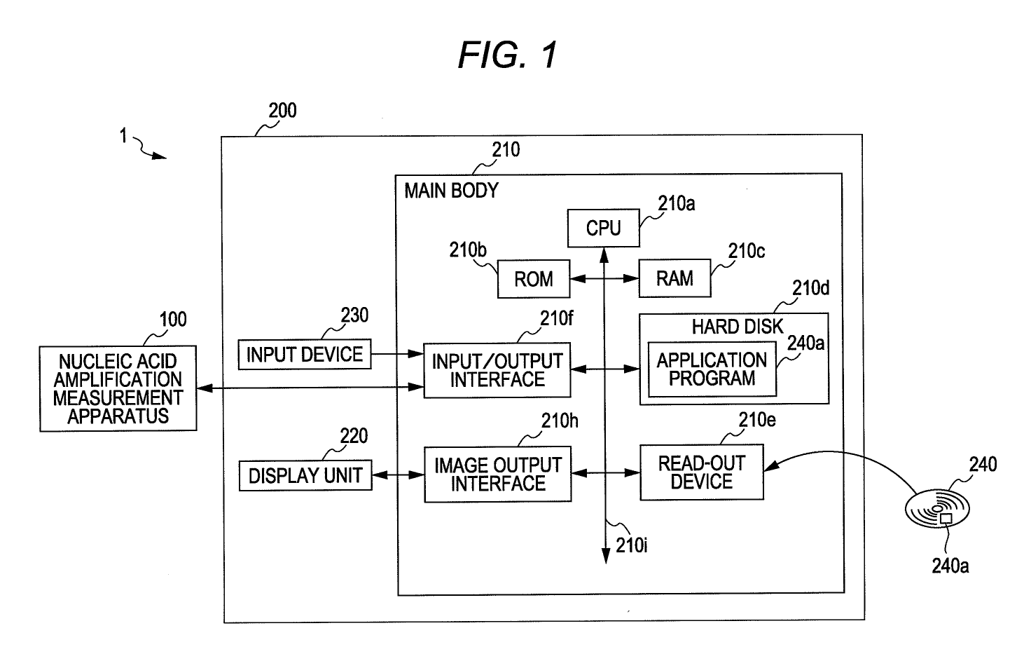

FIG. 1 is a block diagram showing the entire configuration of a determining device 1 according to an embodiment of the present invention.

The determining device 1 according to the present embodiment is configured by a nucleic acid amplification measurement apparatus 100 (hereinafter referred to as “a measurement unit 100”) and an information processing apparatus 200 (hereinafter referred to as “a computer 200”) that is connected to the determining device.

The measurement unit 100 is configured by a PCR device that can execute the method of amplifying nucleic acid such as the real-time RT-PCR assay. The measurement unit 100 amplifies the nucleic acids using mRNAs of SERPINB5, CK15, CK4, PI3, and ANXA8 as templates and measures the fluorescence intensity and the cycle number based on amplification products.

The computer 200 is connected to the measurement unit 100. The computer 200 controls the operation of the measurement unit 100 and determines whether or not lymph node metastasis is positive based on the data of the fluorescence intensity and the data of the cycle number measured by the measurement unit 100.

The computer 200 includes an information processing apparatus main body 210 (hereinafter also referred to as “a determination unit 210”), an input device 230 which inputs necessary data to the determination unit 210, and a display unit (outputting unit) 220 which displays input/output data. An external recording medium 240 is included in the computer 200, if necessary.

The computer 200 is mainly configured by a determination unit 210, a display unit 220, and an input device 230. The determination unit 210 includes a CPU210a, a ROM210b, a RAM210c, a hard disk 210d, a read-out device 210e, an input/output interface 210f, an image output interface 210h, and a bus 210i.

In the determination unit 210, the CPU210a, the ROM210b, the RAM210c, the hard disk 210d, the read-out device 210e, the input/output interface 210f, and the image output interface 210h are respectively connected by the bus 210i so as to mutually transmit and receive data.

The CPU 210a can execute computer programs stored in the ROM 210b and the computer programs loaded in the RAM 210c. A computer program which is executed by the CPU210a and data for the computer program which is executed by the CPU210a are recorded on the ROM210b. The RAM 210c is used to read out the computer programs recorded on the ROM 210b and the hard disc 210d. In executing the computer program, the RAM 210c is used as a work region of the CPU 210a.

The hard disc 210d is installed with various computer programs to be executed by the CPU 210a such as operating system (OS) and application system program, as well as data used in executing the computer program.

The program installed on the hard disk 210d includes an application program 240a which realizes the method of determining lymph node metastasis in cervical cancer and a program which controls the measurement unit 100. As data to be used for executing the program which realizes the method of determining lymph node metastasis in cervical cancer, threshold values or the like are stored in the hard disk 210d.

The read-out device 210e is configured by flexible disc drive, CD-ROM drive, DVD-ROM drive, and the like, and is able to read out computer programs and data recorded on an external recording medium 240. The application program 240a may be recorded on the external recording medium 240, the ROM210b formed in the information processing apparatus main body 210, or the RAM210c.

It is possible that the CPU210a reads out the application program 240a from the external recording medium 240 and the application program 240a is installed on the hard disk 210d.

Operating system providing graphical interface environment such as Windows (registered trademark) manufactured and sold by US Microsoft Co. is installed in the hard disc 210d. In the following description, the application program 240a according to the above-described judgment is assumed to be operating on the operating system.

The input/output interface 210f is configured by serial interfaces such as USB, IEEE 1394, and RS-232C, parallel interfaces such as SCSI, IDE, and IEEE1284, and an analog interface including D/A and A/D converters. The input/output interface 210f is connected to the input device 230 including a keyboard and a mouse. The measurement unit 100 is connected to the input/output interface 210f. Thus, the determination unit 210 can transmit and receive data with the measurement unit 100 via the input/output interface 210f.

The image output interface 210h is connected to the display unit 220 configured by LCD, CRT, or the like, and is configured to output an image signal corresponding to the image data provided from the CPU 210a to the display unit 220. The display unit 220 displays image data (screen) according to the input image signal. The display unit 220 outputs image data provided from the CPU 210a to be described later.

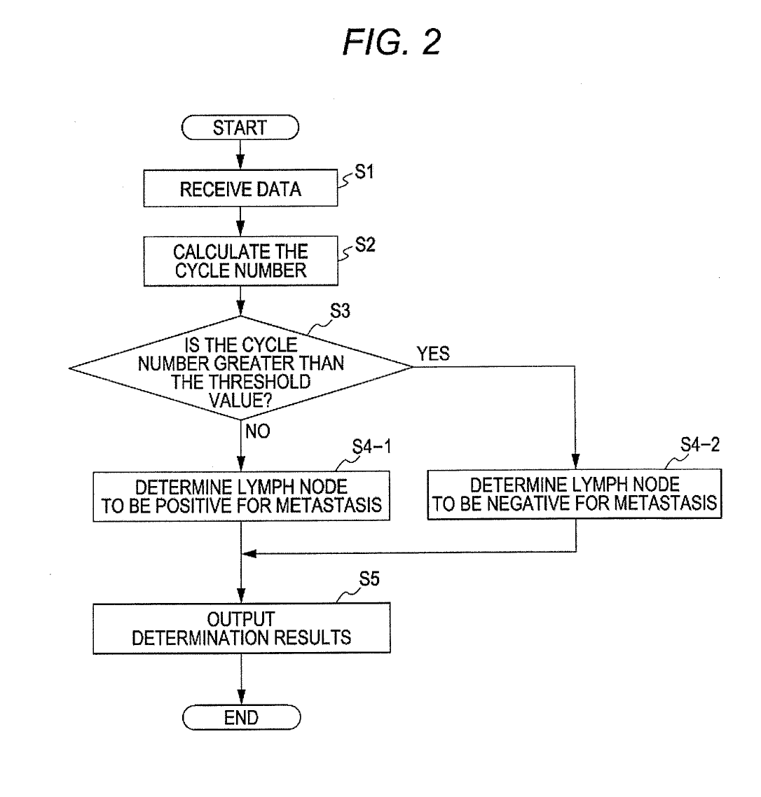

Here, an example of an operation flow of the application program 240a which is executed in the measurement device 1 will be described with reference to FIG. 2.

First, the data of the variation of fluorescence intensity in the nucleic acid amplification reaction and the data of the cycle number are received from the measurement unit 100 via the input/output interface 210f (step S1).

In step S2, the CPU210a calculates the cycle number (cycle number A) when variations of fluorescence intensity in the nucleic acid amplification reaction reach standard values of the genes based on the data of the variation of fluorescence intensity in the nucleic acid amplification reaction and the data of the cycle number. The standard values of the variations in fluorescence intensity when amplifying mRNAs of the genes of SERPINB5, CK15, CK4, PI3, and ANXA8 are 0.04, 0.02, 0.05, 0.02, and 0.03, respectively.

In step S3, the CPU210a compares the data of a threshold value of the cycle number which has been previously stored as the data of the application program 240a in the hard disk 210d with the data of the cycle number A. In step S3, the cycle number A is compared with the threshold value of the cycle number to determine whether it is not more than the threshold value.

When the cycle number A is not more than the threshold value or less (Yes), the process is proceeded to step S4-1. On the contrary, when the cycle number A is not smaller than the threshold value (No), the process is proceeded to step S4-2. The threshold values about the genes are shown in Table 2. The threshold values shown in Table 2 were obtained by calculating an average of Ct values (when the cycle number reached standard values to be described later) of the genes in eight examples of the negative specimens and subtracting 3SD (3 times of standard deviation) from the average.

TABLE 2

| | Threshold value |
|---|---|
| Gene | (Cycle) |
| SERPINB5 | 32.9 |
| CK15 | 32.0 |
| CK4 | 37.7 |
| PI3 | 31.9 |
| ANXA8 | 31.4 |

In step S4-1, the CPU210a determines that cervical cancer has spread to the lymph node tissue (positive for lymph node metastasis). In step S4-2, the CPU210a determines that cervical cancer has not spread to the lymph node tissue (negative for lymph node metastasis).

The CPU210a stores the determination results in the RAM210c and outputs them to the display unit 220 via the image output interface 210h in step S5.

In the present embodiment, the data of the variation of fluorescence intensity in the nucleic acid amplification reaction and the data of the cycle number is acquired from the computer 200 via the input/output interface 210f, however, the present invention is not limited thereto. The cycle number (cycle number A) when the variation of fluorescence intensity in the nucleic acid amplification reaction reaches the standard value may be acquired by inputting the data of the variation of fluorescence intensity in the nucleic acid amplification reaction and the data of the cycle number, from the input device 230, by a user and calculating, from the input values, by the CPU210a.

In the present embodiment, the CPU210a acquires the cycle number A by calculating from the data of the variation of fluorescence intensity in the nucleic acid amplification reaction and the data of the cycle number, however the present invention is not limited thereto. The user may input the cycle number A by the input device 230 and the CPU210a may acquire it.

In the present embodiment, the CPU210a calculates the cycle number A based on the data of the variation of fluorescence intensity in the nucleic acid amplification reaction and the data of the cycle number, however the present invention is not limited thereto. The quantitative value of mRNA of each gene may be calculated from the fluorescence intensity, cycle number, and calibration curve. In this case, the process may be proceeded to step S4-1 when the quantitative value is higher than a predetermined threshold value in step S3, and the process may be proceeded to step S4-2 when the quantitative value is smaller than the predetermined threshold value.

### EXAMPLES

Hereinafter, examples will be described, however, the present invention is not limited thereto.

### Preparation Example 1

A measurement sample was prepared in the following manner using six lymph nodes in which metastasis of cervical cancer was histologically found positive specimens (two lymph nodes positive for adenocarcinoma metastasis and four lymph nodes positive for squamous cell carcinoma metastasis)] and eight lymph nodes in which metastasis of cervical cancer was not histologically found negative specimens (four lymph nodes positive for adenocarcinoma metastasis and four lymph nodes positive for squamous cell carcinoma metastasis)].

First, 4 mL of a pretreatment liquid composition: 200 mM glycine chloride (pH 3.4), 5% by volume of polyoxyethylene lauryl ether (addition mole number of an oxyethylene group: 23) (trade name: Brij (registered trademark) 35), manufactured by Sigma Aldrich), 20% by volume of dimethyl sulfoxide, and 0.05% by volume of an antifoaming agent, trade name: KS-538, manufactured by Shin-Etsu Chemical Co., Ltd.]] was added to the lymph nodes (about 30 to 370 mg/(piece)). Subsequently, the lymph node was homogenized with a blender. The obtained homogenate was centrifuged at 10000×g for 1 minute at room temperature to obtain a supernatant. Then, RNAs were extracted from 400 μL of the supernatant using a kit for RNA extraction/purification (trade name: RNeasy Mini kit, manufactured by QIAGEN, catalog number 74014) and purified them to obtain 60 μL of RNA solution. The absorbance of each of the obtained RNA solutions at a wavelength of 260 nm was measured. The obtained RNA solutions were used as measurement samples.

### Experimental Example 1

Real-time RT-PCR assay was performed by a real-time PCR device (trade name: ABI Prism 7500, manufactured by Applied Biosystems) using the measurement samples obtained in Preparation example 1, a primer pair for SERPIN5B (Maspin), and a quantitative RT-PCR kit (trade name: Quanti Tect SYBR Green RT-PCR kit, manufactured by QIAGEN, catalog number 204245). As for the measurement samples, the cycle number of PCR when the variation of fluorescence intensity based on amplification products reached the standard value was determined. The obtained cycle number of PCR was used as the information about mRNA expression level in the measurement samples (Example 1).

The operation was performed in the same manner as described above except that a primer pair for CK15 (Example 2), a primer pair for ANXA8 (Example 3), PI3 (Example 4) or CK4 (Example 5) was used in place of the primer pair for SERPINB5. As for the measurement samples, the cycle number of PCR when the variation of fluorescence intensity based on amplification products reached the standard value was determined.

Further, the operation was performed in the same manner as described above except that a primer pair for CK19 (Comparative example) or a primer pair for β-actin (Control) was used. As for the measurement samples, the cycle number of PCR when the variation of fluorescence intensity based on amplification products reached the standard value was determined. Here, CK19 is a known molecular marker for lymph node metastasis in cervical cancer. Further, β-actin is a molecular marker known as a housekeeping gene.

The standard values of the variations in fluorescence intensity when amplifying mRNAs of the genes of SERPINB5, CK15, CK4, P13, and ANXA8 are shown in Table 3 below. These standard values indicate the variation of fluorescence intensity when nucleic acid amplification reactions of mRNAs of the genes of SERPINB5, CK15, CK4, PI3, ANXA8, and CK19 are in a logarithmic phase. These standard values are set so as to be equal to the almost same value when converted to the variation of fluorescence intensity per unit chain length.

TABLE 3

| Gene | Standard value |
|---|---|
| SERPINB5 | 0.04 |
| CK15 | 0.02 |
| CK4 | 0.05 |
| PI3 | 0.02 |
| ANXA8 | 0.03 |
| CK19 | 0.07 |

The sequences of primers used for the real-time RT-PCR assay and the composition of the reaction solution are shown in Tables 4 and 5, respectively.

TABLE 4

| | | Sequence | | Sequence |
|---|---|---|---|---|
| Gene | Forward primer | number | Reverse primer | number |
| CK19 | CAGATCGAAGGCCTGAAGGA | 1 | CTTGGCCCCTCAGCGTACT | 2 |
| SERPINB5 | CTTTGTGCTCCTCGCTTGC | 3 | GTTATCCTGGAAAATGCGTGG | 4 |
| CK15 | CTGAAAAGGGTCCCTCGGTC | 5 | AATAGAGCGCATGCAAAGCC | 6 |
| ANXA8 | TCGTTTCAAGGAGCGAGATTG | 7 | CCGCTGGTGTCTTCCATGAT | 8 |
| PI3 | TTAGCCAAACACCTTCCTGACA | 9 | CCGTGACAGCTGCCTCTAGAA | 10 |
| CK4 | CCACTTCCTGCATTTCAGCTT | 11 | CACCACCTCCAGCAAAAACC | 12 |

TABLE 5

| | Volume (μL) |
|---|---|
| RNase-free water | 10.95 |
| 2 x QuantiTect SYBR Green RT-PCR | 12.50 |
| Master Mix *¹ (Product name) | |
| 100 nM forward-primer solution | 0.15 (Final |
| | concentration 600 μM) |
| 100 nM riverse-primer solution | 0.15 (Final |
| | concentration 600 μM) |
| Quanti Tect RT Mix *² (Product | 0.25 |
| name) | |
| Measurement sample | 1.00 |
| Total | 25.00 |

The marks *¹ and *² indicate reagents included in Quantitative RT-PCR kit

Thermal profiles in the real-time RT-PCR assay show that a cycle of keeping the measurement sample warm at 50° C. for 30 minutes, keeping it warm at 95° C. for 10 minutes, denaturalizing it at 94° C. for 15 seconds, annealing it at 53° C. for 30 seconds, and elongating it at 72° C. for 30 seconds was carried out 40 times.

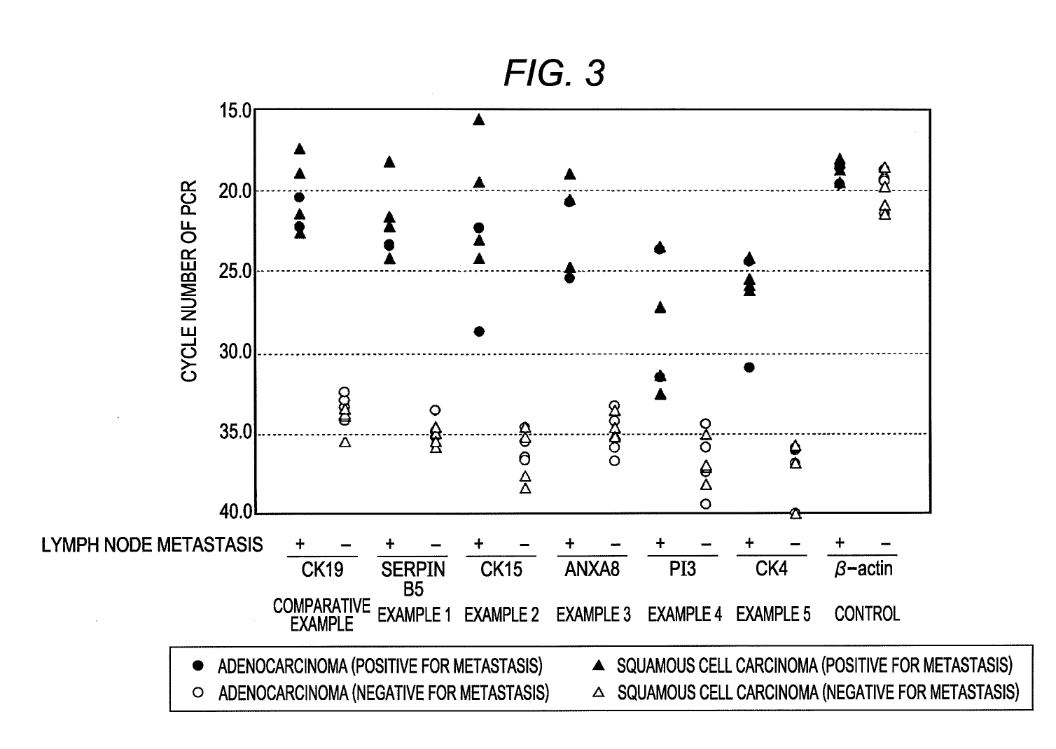

FIG. 3 shows a relationship between the presence of lymph node metastasis as for the genes and the PCR cycle number which is obtained from the results of Experimental example 1. In FIG. 3, “the PCR cycle number” represents the cycle number when the variation of fluorescence intensity derived from amplification products reach each of the standard values in the real-time RT-PCR assay using the measurement sample of the positive specimen or the measurement sample of the negative specimen. The “+” mark indicates the cycle number of PCR when the measurement sample of the positive specimen is used and the “−” mark indicates the cycle number of PCR when the measurement sample of the negative specimen is used.

FIG. 3 shows that CK19 (Comparative example), SERPINB5 (Example 1), CK15 (Example 2), ANXA8 (Example 3), PI3 (Example 4), and CK4 (Example 5) have clear differences in the PCR cycle numbers among the positive specimens and the negative specimens. More specifically, it is found that the PCR cycle numbers of the negative specimens are less than the PCR cycle numbers of the positive specimens. That is, it is found that the mRNA expression levels of SERPINB5, CK15, CK4, P13, and ANXA8 in the lymph node tissues in which cervical cancer has spread are higher than those in the lymph node tissues in which cervical cancer has not spread. On the other hand, there is almost no difference in the PCR cycle numbers among the positive and negative specimens of β-actin (housekeeping gene). That is, it is found that there is almost no difference in the mRNA expression levels of β-actin among the positive and negative specimens. As is clear from the results, it is determined whether cervical cancer has spread to the lymph node tissue based on mRNA expression level of at least one gene selected from SERPINB5, CK15, CK4, PI3, and ANXA8.

From FIG. 3, it is found that there are clear differences in the PCR cycle numbers among the positive specimens and negative specimens of SERPINB5, CK15, CK4, PI3, and ANXA8, in both cases between cervical adenocarcinoma and cervical squamous cell carcinoma, More specifically, the positive specimens can be distinguished from the negative specimens by setting the threshold values of the PCR cycle numbers of SERPINB5, CK15, CK4, PI3, and ANXA8 to 32.9, 32.0, 37.7, 31.9, and 31.4 respectively. This shows that the metastasis to the lymph node tissue can be determined based on the mRNA expression levels of SERPINB5, CK15, CK4, PI3, and ANXA8 regardless of the histological type of cervical cancer.

From FIG. 3, it is found that differences in the PCR cycle numbers among the positive and negative specimens of SERPINB5 (Example 1) and CK15 (Example 2) are equal to those of CK19 (Comparative example). This shows that it is determined whether cervical cancer has spread to the lymph node tissue with high accuracy by using the mRNA expression level of, particularly of the SERPINB5 or CK15 gene.

What is claimed is:

1. A method of determining lymph node metastasis in cervical cancer, comprising:
acquiring information about mRNA expression level of at least one gene selected from the group consisting of SERPINB5, CK15, CK4, PI3, and ANXA8 contained in a measurement sample prepared from a lymph node tissue from a cervical cancer patient which may have cervical cancer metastasis; and
determining whether cervical cancer has spread to the lymph node tissue based on the acquired information about mRNA expression level.

2. The method according to claim 1, wherein the determination process determines that when the mRNA expression level is determined to be overexpressed from the information about mRNA expression level, cervical cancer has spread to the lymph node tissue.

3. The method according to claim 1, wherein the information about mRNA expression level is acquired by a method of amplifying nucleic acid.

4. The method according to claim 3, wherein the method of amplifying nucleic acid is a polymerase-chain-reaction assay, a strand displacement activity assay, a ligase-chain-reaction assay or a transcriptional amplification method.

5. The method according to claim 3, wherein the information about mRNA expression level is a value based on the cycle number of nucleic acid amplification reaction by the method of amplifying nucleic acid.

6. The method according to claim 5, wherein the determination process compares the cycle number of nucleic acid amplification reaction with a predetermined threshold value and determines that cervical cancer has spread to the lymph node tissue when the cycle number is smaller than the predetermined threshold value.

7. The method according to claim 6, wherein the determination process further determines that cervical cancer has not spread to the lymph node tissue when the cycle number of nucleic acid amplification reaction is larger than the predetermined threshold value.

8. The method according to claim 3, wherein the information about mRNA expression level is a quantitative value of mRNA which is calculated by the method of amplifying nucleic acid.

9. The method according to claim 8, wherein the quantitative value of mRNA is compared with a predetermined threshold value and it is determined that cervical cancer has spread to the lymph node tissue when the quantitative value is larger than the predetermined threshold value.

10. The method according to claim 9, wherein it is further determined that cervical cancer has not spread to the lymph node tissue when the quantitative value of mRNA is smaller than the predetermined threshold value.

11. The method according to claim 1, wherein the lymph node tissue is a tissue containing a pelvic lymph node.

12. The method according to claim 11, wherein the pelvic lymph node is at least one selected from the group consisting of common iliac lymph nodes, external iliac lymph nodes, internal iliac lymph nodes, middle rectal root lymph nodes, and obturator lymph nodes.

13. The method according to claim 1, wherein the measurement sample prepared from the lymph node tissue is a sample obtained by homogenizing the lymph node tissue which may have cervical cancer metastasis in the pretreatment liquid.

14. The method according to claim 13, wherein a pH of the pretreatment liquid is from 2.5 to 5.0.

15. The method according to claim 13, wherein the pretreatment liquid contains a buffer.

16. The method according to claim 13, wherein the pretreatment liquid contains a surfactant.

17. A device for determining lymph node metastasis in cervical cancer, comprising:
a measurement unit which acquires information about mRNA expression level of at least one gene selected from the group consisting of SERPINB5, CK15, CK4, PI3, and ANXA8 contained in a measurement sample prepared from a lymph node tissue which may have cervical cancer metastasis;
a determination unit which determines whether cervical cancer has spread to the lymph node tissue based on the information about mRNA expression level acquired by the measurement unit; and
an output unit which outputs the determination result obtained by the determination unit.

18. A computer program product comprising: a computer readable medium; and software instructions on the computer readable medium, for enabling the computer to perform predetermined operations comprising:
receiving information about mRNA expression level of at least one gene selected from the group consisting of SERPINB5, CK15, CK4, PI3, and ANXA8 contained in a measurement sample prepared from a lymph node tissue which may have cervical cancer metastasis;
determining whether cervical cancer has spread to the lymph node tissue based on the information about mRNA expression level received in the receiving step; and
outputting the determination result obtained by the determining process.
